(19) United States
(12) Patent Application Publication
(10) Pub. No.: US 2026/0263705 A1
(43) Pub. Date: Sep. 10, 2026

(54) MEDICAMENT DELIVERY DEVICE

(71) Applicant: SHL Medical AG, Zug (CH)

(72) Inventors: Meng-Jhen Chiou, Taoyuan City (TW); Tzu-Chia Lee, Taoyuan City (TW); Pei-Hsin Liu, Taoyuan City (TW); Chiao-Yi Chiu, Taoyuan City (TW); Pei Yu Chao, Taoyuan City (TW); Yun Hsuan Chang, Taoyuan (TW)

(21) Appl. No.: 19/163,922

(22) PCT Filed: Mar. 4, 2024
(86) PCT No.: PCT/EP2024/055590

§ 371 Date: Sep. 10, 2025

(30) Foreign Application Priority Data
Mar. 14, 2023 (EP) 23161896.8

(51) Int. Cl. A61M 5/32; A61M 5/20

(52) U.S. Cl. CPC A61M 5/3243; A61M 5/2033

(57) ABSTRACT

The present disclosure provides a medicament delivery device including a housing body with a generally tubular shape configured to hold a medicament delivery system and a drive assembly. The medicament delivery device further includes a delivery member cover configured to cover at least a proximal portion of the medicament delivery system. The delivery member cover is further configured to move, when pressed against tissue at an injection site, coaxially within the housing body from a first proximal position to at least a second more distal position The delivery member cover is further configured to activate by movement from the first position towards the second position the drive assembly to initiate medicament delivery. A proximal end portion of the housing body comprises a flexible structure, an annular flange is provided at a proximal end of the flexible structure, and the annular flange is configured to at least partially surround the delivery member cover and to prevent contact between side surfaces of the device and the tissue of the injection site during use of the device.

### TECHNICAL FIELD

The present invention relates to a medicament delivery device for obese patients.

### BACKGROUND

Many medicament delivery devices are developed for self-administration, i.e., a user performs the medicament delivery her-, or himself. This requires a medicament delivery device, which is as safe to use and as easy to handle as possible.

In order to meet these requirements, the risk of human errors must be minimized, the number of actions needed to be performed in order to receive a dose need to be reduced and the device must be intuitive and ergonomic to use. Thus, in order to minimize the risk of human errors, it is desirable to have the device as pre-assembled as possible.

Medicament delivery devices, such as autoinjectors and manual injectors, often have a needle shield or needle cover that extends in the proximal direction from the housing. The needle shield protects the needle from exposure before medicament delivery. The needle shield may be arranged to move along the longitudinal axis of the housing in a distal direction further into the housing, when the medicament delivery device is pushed towards an injection site. The needle is thus exposed so that it can penetrate the patient's skin. For autoinjectors, this movement of the needle shield may cause the medicament delivery device to become activated to discharge medicament via the needle.

US 2014/0114247 A1 relates to an injection device comprising a housing, a container holder arranged within said housing, the container holder being configured for accommodating a medicament container having a needle attached to one end thereof and a stopper sealingly and slidable arranged inside the medicament container at the other end thereof, a drive unit comprising a plunger rod and plunger drive means, the plunger drive means being slidable arranged in relation to the plunger rod, being rotationally locked to the plunger rod and being rotatable in relation to the housing.

US 2019/0022334 A1 relates to a safety device for a pre-filled syringe with an injection needle comprising a support body adapted to mount the pre-filled syringe, a needle shield slidably arranged with respect to the support body, a retention and locking means for retaining and locking the needle shield with respect to the support body in a first and a second advanced position and in a retracted position. The needle shield comprises an annular flange adapted to rest on the skin of the patient receiving an injection.

EP 3 007 752 A1 relates to an injection device having a contact surface for pressing against the skin of a patient. The contact surface comprises an inwardly extending or radially extending flange or be at the end of a tapered or flared portion of the sliding sleeve to provide an improved contact area.

U.S. Pat. No. 10398854 B2 relates to an injector such as a needle-type autoinjector having a flexible flange disposed at an injection end of the injector for stretching or pinching the skin of the injection site. The flexible flange may be removably attached to the injector or integrated into the injector during manufacturing.

### SUMMARY

Many medicament delivery devices are developed for self-administration. However, if given incorrectly, intramuscular injections may result in poor absorption of drug, reduced drug effectiveness, or irritation to surrounding tissues.

Medicament delivery devices of the types mentioned above, on the market today, do generally not take obese patient skin conditions into account in their designs. For obese patients the amount of subcutaneous tissue present, the weight of the patient, muscle mass, and subcutaneous fat at the injection site may be different. That is, the tissue at the injection site may be softer compared to a normal-weight patient due to the different fat to muscle ratio and thickness of the tissue.

Furthermore, during medicament delivery, the soft skin may fold around the at least part of medicament delivery device and the interaction between the medicament delivery device with the folding skin in obese patients may create friction, which results in that a higher activation force is required.

In other words, a larger force is required to push the needle shield into the housing. Some patients with obesity may suffer from impaired dexterity and/or reduced strength, making it challenging to administer medication.

The above problems contribute to a different resistance of the skin against the activation mechanism of the medicament delivery device. Thus, the medicament delivery device may not be activated correctly.

On the other hand, if more pressure is applied to obtain more resistance for activation, stretching or bunching of the skin during injection is possible.

With the increasing rates of obesity, caution needs to be taken to determine the amount of subcutaneous fat of the tissue at the injection site, to select the correct needle length, to insert at a predefined angle, and to flatten the skin rather than to bunch the skin.

As mentioned above, a medicament delivery device generally comprises a housing body configured to receive and/or hold the main functional components: an activation system, a medicament delivery system, and a drive assembly.

Furthermore, the housing body may be configured to receive a cap to cover a proximal end portion of the medicament delivery device. The cap is usually removed prior to use of the device.

Many examples of a medicament delivery system are known. Examples are a prefilled syringe based system or a cartridge based system. A medicament delivery system generally comprises a delivery member configured to deliver an injection into the tissue. A typical delivery member is a needle.

In order to deliver an injection, the medicament delivery system generally requires energy. Said energy is provided by the drive assembly. Many examples of a drive assembly are known. Generally, a drive assembly comprises a driver and an energy storage member. In some disclosures the energy storage member is referred to as power pack assembly. In one example the energy storage member is a compression or torsion spring; the driver is moved in a proximal direction and coupled to a plunger of a syringe.

Generally, an activation system is configured to activate the injection, i.e. to affect an energy release from the drive assembly to the medicament delivery system. An example the activation system is a push button at the distal end of the housing body configured to activate the drive assembly.

The present disclosure does not provide a solution for the medicament delivery system, activation system, and/or the drive assembly.

An object of the present disclosure is to provide a medicament delivery device which solves, or at least mitigates, problems of the prior art. The above problems are among those solved by the present disclosure as defined in the appended claims.

In an embodiment of the present disclosure, a medicament delivery device comprises a housing body with a generally tubular shape configured to hold a medicament delivery system and a drive assembly; and a delivery member cover: configured to cover at least a proximal portion of the medicament delivery system, configured to move, when pressed against tissue at an injection site, coaxially within the housing body from a first proximal position to at least a second more distal position, and further configured to activate by movement from the first position towards the second position the drive assembly to initiate medicament delivery. Wherein a proximal end portion of the housing body comprises a flexible structure; an annular flange is provided at a proximal end of the flexible structure; and the annular flange is configured to at least partially surround the delivery member cover and to prevent contact between side surfaces of the device and the tissue of the injection site during use of the device.

In an embodiment of the present disclosure, the flexible structure is configured to be compressible in a direction of a proximal-to-distal axis of the housing body.

In an embodiment of the present disclosure, the flexible structure has an axial through opening in a direction of the proximal-to-distal axis of the housing body and is configured to allow the delivery member cover to move within the flexible structure.

In an embodiment of the present disclosure, the inner diameter of the through opening is at least the outer diameter of the delivery member cover.

In an embodiment of the present disclosure, when the flexible structure is in a relaxed state the annular flange is essentially flush with the proximal end of the delivery member cover in the first position; and when the flexible structure is in a compressed state the annular flange is essentially flush with the proximal end of the delivery member cover in the second position.

In an embodiment of the present disclosure, the annular flange is configured to close a gap between an inner surface of the flexible structure and an outer surface of the delivery member cover.

In an embodiment of the present disclosure, a medicament delivery device further comprises a cap, wherein the delivery member cover further comprises cap engagement means configured to engage with corresponding means on an inner surface of the cap; and wherein the cap is configured to cover at least the flexible structure of the housing body when the cap engagement means are engaged.

In an embodiment of the present disclosure, the flexible structure is spring structure.

In an embodiment of the present disclosure, the spring structure is one of a helical spring, a compression spring, a disk spring, a variable rate spring, a wave spring, a curved spring, a Belleville spring, or a finger spring.

Based on numerical studies and experimental tests the applicant has realized that the main factor for the optimization of the activation force for obese patients is the ratio between the outer diameter of the housing and the outer diameter of the needle cover as this factor directly influences the amount of contact between parts of the device and the skin of the injection site.

A further factor is the internal resistance force of the activation mechanism, e.g., the spring force of the needle cover. A lower spring force will reduce the aforementioned problems; however, the optimization is limited because a certain spring force is required to ensure the device functionality as well as to fulfill industry requirements for medicament delivery devices. Thus, in this disclosure this factor is not optimized.

Still a further factor is the offset at full stroke position. The latter being the distance between the contacting surface of the proximal end of the needle cover and the end portion of the housing. It has been found that increasing this distance lowers the activation force. However, at some offset distance, the activation force on soft tissue will plateau due to less resistance because the soft tissue will have less contact with the housing. Thus, in this disclosure this factor is not optimized.

It has further been realized that all three factors may be interrelated, however, to a certain extend they can be independently improved.

It is a first general concept of the disclosure, to provide an extended surface area at the injection site, i.e., the proximal end portion of the needle cover. This is illustrated in FIG. 3.

FIG. 3 illustrates the results of a simulation of an injection with different Models of injectors into obese skin 10. In the columns of FIG. 3 results of four different Models of embodiments of the disclosure are shown. The Models differ in the respective amount of diameter extension. In the rows of FIG. 3 two different amounts of force are applied. All figures show results for the same tissue.

Model #1 has no diameter extension and ratio of 0.79. That is, the outer diameter of the needle cover is smaller than the inner diameter of the housing body.

Model #2 has a diameter extension and a ratio of 0.92. Model 3 has a ratio of 1. Model 4 has a ratio of 1.9.

The simulations show that an increased diameter portion has two main effects.

First, it increases the contact surface with the skin 10 and thus reduces the local pressure on the tissue, which allows for more movement in the activation mechanism of the device at the same pressure. This is evident from a comparison at the solid line P1 of the first row. The needle cover of Model #1 has moved the least and the needle cover of Model #4 has moved the most. However, the needle covers of Model #3 and Model #4 have moved almost the same.

Second, the shape of the folding tissue 10 is changed to an extent that the contact between the skin of the injection site and the moving end portion during activation, i.e., needle shield and/or housing body, is reduced or prevented.

This is evident from the second row of FIG. 3 at the solid line P2. The second row shows a subsequent movement of the housing body 1 during activation. While the needle covers in Model #3 and Model #4 have already reached the respective end position, little to none contact between the tissue 10 and on the side surfaces of the housing body 1 is observed. In Contrast the devices according to Model #1 and Model #2 still require more pressure to reach the end position and exhibit a substantial amount of contact between the tissue 10 at the side surfaces of the housing body.

Thus, a ratio of about 1 between the outer diameter of the needle shield 2 and the housing body 1 is a preferred ratio for embodiments of this disclosure. A smaller ratio still allows for some contact with the tissue during activation and a higher ratio does not further optimize the activation force.

The optimum ratio was determined for an essentially flat and circular contact surface with the tissue 10. However, other shapes of contact surfaces are possible. Concave, convex, or complex axial shapes and/or elliptical or complex radial shapes are possible. For said shapes, a comparable ratio is obtained, however, for small deviations from a circular shape the overall effect will be the same.

If the ratio is about 1 and the outer diameter of the housing body is essentially the same as the diameter of the extended surface area portion, the activation force is optimized and the contact with the tissue (except for the intended injection site contact area) is minimized.

It is a second general concept of the disclosure to provide an additional cover, preferably in form of a sleeve, covering a proximal end portion of the needle cover and/or the housing body and thus reduce or prevent the contact with the tissue at the injection site during activation. This is illustrated in FIG. 4.

FIG. 4 shows three different embodiments of the disclosure at the same push pressure. The embodiments on the left, Model #5 and Model #5b are similar to Model #1 and Model #3 as discussed before, respectively. The embodiments of Model #5a has a ratio of about 1. The embodiment Model #6 also has a ratio of about 1, thus it also corresponds to Model #3 as discussed above, but additionally comprises a sleeve portion projecting distally from contact surface. In Model #5a and Model #5b the tissue is in contact with the side surface of the needle cover and the movement of the housing body 1 is not completed, see the two solid lines and the distance A therebetween.

In Model #6, at the same pressure, the skin is only in contact with the sleeve portion and the movement is already completed. Thus, the provision of a sleeve portion further optimizes the activation force.

It is a third general inventive concept of this disclosure, to provide a moving or sliding cover, which comes in contact with the tissue, but does reduce the friction, because the skin is not in contact with the activation mechanism.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings, in which

FIG. 1A shows a cross sectional side view of a device according to an embodiment of the present disclosure.

FIG. 1B shows a side view of the device according to the embodiment of the present disclosure with a transparent cap.

FIG. 1C shows a perspective view of a proximal end portion of the device according to the embodiment of the present disclosure with a removed cap.

FIG. 2A shows a perspective view of a medicament delivery device.

FIG. 2B shows a perspective view of a proximal end portion of the device of FIG. 2A with a removed cap.

FIG. 3 shows results of a numerical simulation of the activation for different embodiments of the present disclosure.

FIG. 4 shows results of a numerical simulation of the activation for different embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

For the purpose of the disclosure the pre-activation is defined as the button is not pressed, and the used pushes the device against their abdomen first. During pre-activation a force is required to bring the device into the first position.

The medicament delivery device of embodiments according to the present disclosure may comprise a housing 1 configured to house the components of a medicament delivery device. The housing may be tubular. The housing may extend along an axis L, which is the longitudinal axis of the tubular shaped housing. The housing has a proximal end and a distal end. The housing has a body which extends between the proximal end and the distal end.

The medicament delivery device may further comprise a delivery member cover, also referred to as needle shield. The delivery member cover may be generally tubular or tubular. The delivery member cover is arranged in the housing and extends proximally from a proximal opening of the housing.

The delivery member cover may be configured to be moved linearly relative to the housing along the axis L. The delivery member cover is configured to be moved linearly from an extended position to a retracted position relative to the housing. In the retracted position the delivery member cover is received further by the housing.

The medicament delivery device may further comprise a drive assembly (not shown) arranged in the housing. The drive assembly may be any drive assembly used in an autoinjector or a manual injector which activates or triggers medicament delivery by movement of the delivery member cover from the extended position towards the retracted position. Examples of suitable drive assemblies are for example disclosed in WO2011/123024, WO2019/011688, WO2019/011689, WO2019/011690, and WO2019/063267, incorporated herein by reference.

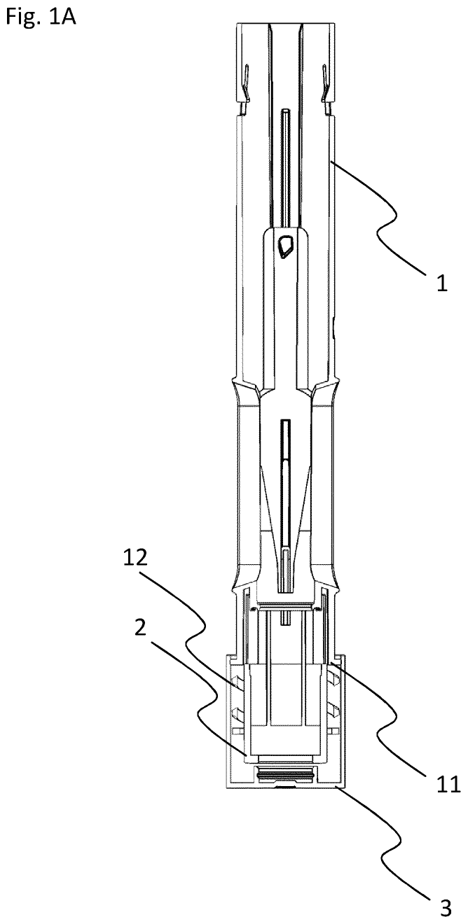

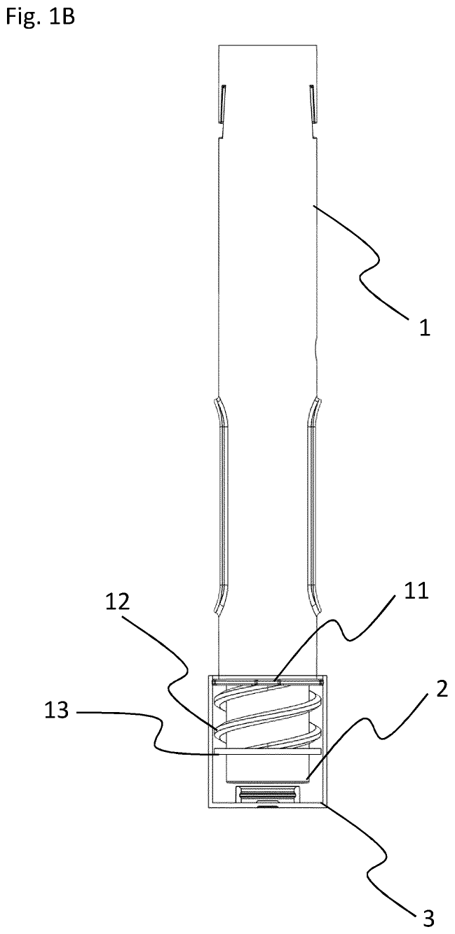

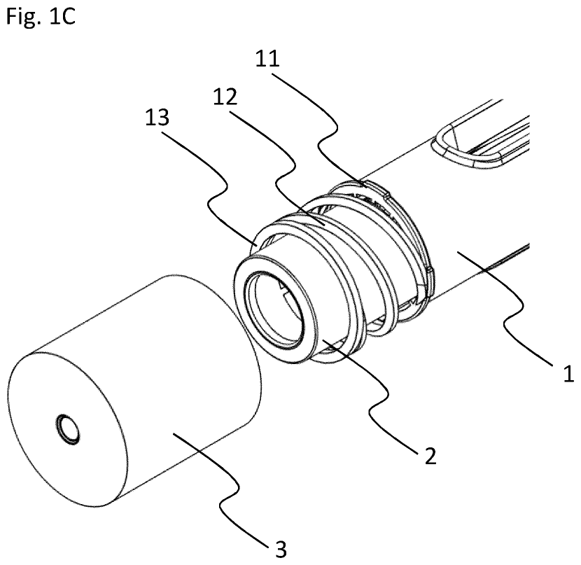

FIGS. 1A to 1C illustrate an embodiment of the present invention. FIG. 1A shows a cross sectional side view of a device according to an embodiment of the present disclosure. FIG. 1B shows a side view of the device according to the embodiment of the present disclosure with a transparent cap. FIG. 1C shows a perspective view of a proximal end portion of the device according to the embodiment of the present disclosure with a removed cap.

In a preferred embodiment the housing 1 comprises at a proximal end portion a flexible structure 12.

It is a first general concept of the disclosure, that the flexible structure 12 of the housing 1 does not move along the axis L, compared to the range of movement of the needle cover 2 and the housing 1, during the activation. Thus, even if the flexible structure contacts folding tissue, little to no friction occurs and the activation force is reduced.

It is a second general concept of the disclosure, that an end portion of the flexible structure 12 may be configured as diameter extension as discussed above. Thus, the contact between the tissue and the side wall of the housing, i.e., the flexible structure 12, is further reduced.

In this disclosure the term “configured to prevent contact” is defined as to be formed such that during the activation movement of the needle cover 2 and the corresponding movement of the housing 1 a contact between the tissue folding around the proximal end surface (see above) and any side surface, i.e. of the needle cover 2 and/or housing 1, is essentially prevented or reduced such that the movement is not significantly biased by the friction between the tissue and the contact surface.

Therefore, the flexible structure is configured to be compressible in a direction of the axis L. An additional compression in a different radial direction may also occur.

In a preferred embodiment the flexible structure 12 is integrally formed in the proximal end portion of the housing 1.

Alternatively, the flexible structure may be a separate part configured to be connected to the proximal end portion of the housing 1 either via a mechanical connection or my means of and adhesive or bonding method. In case of a mechanical connection the flexible structure 12 and/or the end portion of the housing may comprise suitable connection means (not shown).

In a preferred embodiment, the flexible structure 12 is configured to surround at least a proximal end portion of the needle cover 2. Therefore, the flexible structure comprises a through opening in the axial direction. The minimal inner diameter of the through opening is at least the diameter of the needle cover 2.

In a preferred embodiment the flexible structure comprises at the proximal end portion an annular flange 13. The annular flange 13 is configured to surround a proximal end portion of the needle cover 2.

That is, in a preferred embodiment, the annular flange 13 is configured to bridge a gap between the outer surface of the needle cover and the inner surface of the flexible structure 12.

In other words, the inner diameter of the annular flange 12 corresponds to the outer diameter of the proximal end portion of the needle cover 2. The outer diameter of the annular flange portion is preferably essentially the same as the outer diameter of the proximal end portion of the housing, i.e., the outer diameter of flexible structure 12.

In a preferred embodiment the annular flange 13 is integrally formed in the proximal end portion of the flexible structure 12.

Alternatively, the annular flange 13 may be a separate part configured to be connected to the proximal end portion of the flexible structure 12 either via a mechanical connection or my means of and adhesive or bonding method. In case of a mechanical connection the annular flange 13 and/or the end portion of the flexible structure 12 may comprise suitable connection means (not shown).

The flexible structure 12 is configured to allow a movement of the annular flange 13 from a proximal position to a distal position corresponding to the movement of the needle cover 2 from a first to a second position during the activation.

That is, when the device is pressed against the tissue of an injection site, the annular flange 13 forms together with the proximal end surface of the needle cover 2 a contact area. As discussed above, during the activation movement, the flexible structure 12 is compressed and the annular flange 13 moves distally together with the needle cover 2.

During this movement the flexible structure 12 prevents contact between the tissue and the needle cover 2 and also between the tissue and the housing 1. It is noted that the flexible structure 12 is a part of the housing 1 and it may come in contact with the folding tissue. However, the flexible structure 12 itself is merely compressed and not moved during the movement of the needle cover 2 and the remainder of the housing 1. Thus, contact between a moving portion of the housing 1 is prevented.

Furthermore, in a preferred embodiment the outer diameter of the annular flange 13 is essentially the same as the outer diameter of the housing 1.

That is, when the flexible structure 12 is integrally formed with the housing 1, the diameter of the flexible structure is preferably the same as the diameter of the remainder of the housing. Thus, as described above, because of the optimized ratio of about 1, the contact is further reduced and/or prevented.

In a preferred embodiment the housing 1 further comprises cap engagement means 11 formed at an outer surface of a proximal end portion of the housing 1. Preferably, the cap engagement means 11 are provided distally to the flexible structure 12. The cap engagement means 11 are configured to engage with corresponding means on a cap 3.

In a preferred embodiment, the cap engagement means 11 are sections, preferably four sections, of an annular flange formed at the distal end of the flexible structure 12. Accordingly, the cap 3 comprises corresponding recesses, preferably a circular recess formed at a distal end portion of the inner surface of the cap 3. This way, the cap 3 can be attached in any rotational orientation.

In preferred embodiment, the flexible structure 12 is preferably formed as a spring structure. The spring structure 12 is preferably formed integrally with the proximal end portion of the housing 1.

The spring structure may be any suitable spring structure with a hollow core for accommodating the needle cover 2. Suitable spring structures are, e.g., helical spring, compression spring, disk spring, variable rate springs, wave spring, curved spring, Belleville spring, and finger spring.

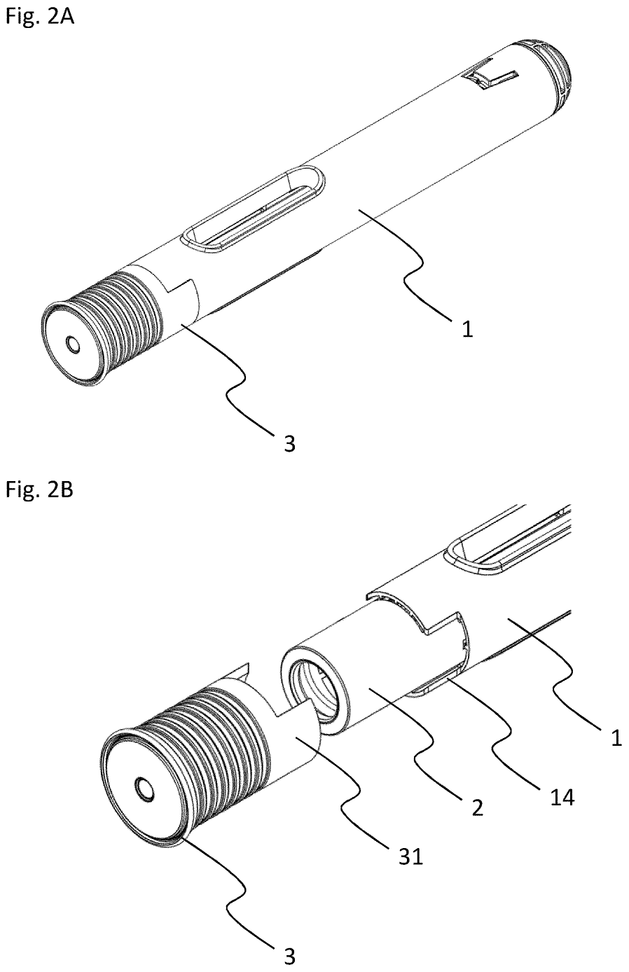

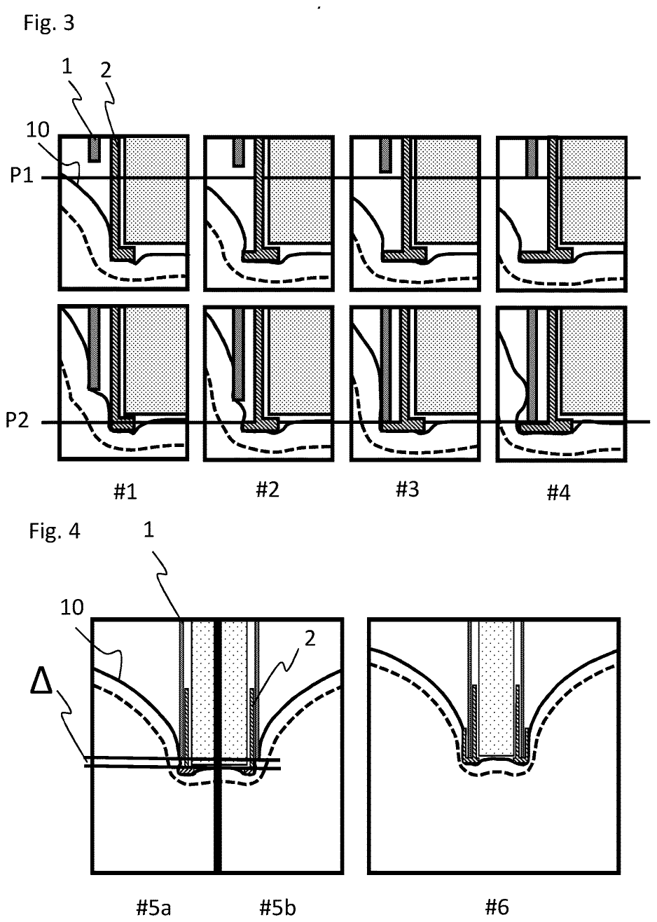

As discussed above, the activation force is also reduced, when contact between the moving housing 1 and the tissue is reduced or prevented. Thus, in a further embodiment of the present disclosure, as illustrated in FIG. 2A and FIG. 2B, the housing 1 comprises two or more cut outs 14 in the distal end portion. The sections between the cut-outs 14 form protrusions. Two is a preferred number of cut-outs 14.

Due to the cut-outs, the tissue may only contact the protrusions but may not contact the needle cover 2 in the cut outs 14. Thus, compared to a device without cut-outs, the contact and thus the friction is effectively reduced.

A corresponding cap 3 may comprise corresponding flaps 31 protruding distally from the cap main body, The flaps 31 correspond to the cut outs 14 in shape and size such that when the cap 3 is attached to the device, the needle cover 2 is protected.

Furthermore, the needle cover 2 and/or the housing 1 may comprise one or more cap engagement means 11, configured to hold the cap 3 on the proximal end portion of the housing 1.

Additionally or alternatively the cap 3 may comprise an inner structure, e.g., a rigid needle shield, RNS, remover.

In a preferred embodiment the movement of the needle cover 2 is blocked by one or more components of the cap 3. That is, the device cannot be activated while the cap 3 is attached to the housing 1 and/or needle cover 2.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g., an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC 500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Various modifications to the embodiments described are possible and will occur to those skilled in the art without departing from the invention which is defined by the following claims.

1-9. (canceled)

10. A medicament delivery device comprising
a housing body with a generally tubular shape configured to hold a medicament delivery system and a drive assembly; and
a delivery member cover:
configured to cover at least a proximal portion of the medicament delivery system,
configured to move, when pressed against tissue at an injection site, coaxially within the housing body from a first proximal position to at least a second more distal position, and
further configured to activate by movement from the first position towards the second position the drive assembly to initiate medicament delivery; and
wherein a proximal end portion of the housing body comprises a flexible structure,
wherein an annular flange is provided at a proximal end of the flexible structure, and
wherein the annular flange is configured to at least partially surround the delivery member cover and to prevent contact between side surfaces of the device and the tissue of the injection site during use of the device.

11. The medicament delivery device according to claim 10,
wherein the flexible structure is configured to be compressible in a direction of a proximal-to-distal axis of the housing body.

12. The medicament delivery device according to claim 10,
wherein the flexible structure has an axial through opening in a direction of the proximal-to-distal axis of the housing body and is configured to allow the delivery member cover to move within the flexible structure.

13. The medicament delivery device according to claim 12,
wherein the inner diameter of the through opening is at least the outer diameter of the delivery member cover.

14. The medicament delivery device according to claim 11,
wherein when the flexible structure is in a relaxed state the annular flange is essentially flush with the proximal end of the delivery member cover in the first position; and
wherein when the flexible structure is in a compressed state the annular flange is essentially flush with the proximal end of the delivery member cover in the second position.

15. The medicament delivery device according to claim 10,
wherein the annular flange is configured to close a gap between an inner surface of the flexible structure and an outer surface of the delivery member cover.

16. The medicament delivery device according to claim 10, further comprising a cap,
wherein the delivery member cover further comprises cap engagement means configured to engage with corresponding means on an inner surface of the cap; and
wherein the cap is configured to cover at least the flexible structure of the housing body when the cap engagement means are engaged.

17. The medicament delivery device according to claim 10,
wherein the flexible structure is a spring structure.

18. The medicament delivery device according to claim 17,
wherein the spring structure is one of a helical spring, a compression spring, a disk spring, a variable rate spring, a wave spring, a curved spring, a Belleville spring, or a finger spring.
